(19) United States
(12) Patent Application Publication
(10) Pub. No.: US 2025/0111915 A1
(43) Pub. Date: Apr. 3, 2025

(54) System for Reviewing Patient Data from Remote Patient Monitoring Devices

(71) Applicants: Stanley G. Van Meter, II, Lake Mary, FL (US); Matt Furnari, Mount Dora, FL (US)

(72) Inventors: Stanley G. Van Meter, II, Lake Mary, FL (US); Matt Furnari, Mount Dora, FL (US)

(21) Appl. No.: 18/969,524

(22) Filed: Dec. 5, 2024

Related U.S. Application Data
(63) Continuation of application No. 17/243,392, filed on Apr. 28, 2021.

(51) Int. Cl. G16H 15/00; A61B 5/00; G06F 3/04883; G06F 21/62; G06N 20/00; G16H 10/60; G16H 80/00

(52) U.S. Cl. CPC G16H 15/00; A61B 5/0022; A61B 5/7435; A61B 5/7475; G06F 3/04883; G06F 21/6254; G06N 20/00; G16H 10/60; G16H 80/00

(57) ABSTRACT

Data is collected from any number of Remote Patient Monitoring devices and is stored in a HIPAA certified database and can be linked to a person. To analyze the data, the links to the patients are anonymized so that the data has no Personally Identifiable Information (PII). This data is placed in a database in a manner that allows a group of medical analysts to select any number of records to analyze. Each record is analyzed by Artificial Intelligence (AI). Both AI and human analyst data are stored with the anonymized record. If the results match, records are transmitted back to the original database and rejoined with the patient data. If the results do not match, they are sent to another database where another medical analyst can review the data. After a second human review, the results are transmitted back to the original database and rejoined with the patient data.

FEDERALLY SPONSORED RESEARCH: Not Applicable

SEQUENCE LISTING OR PROGRAM: Not Applicable

### TECHNICAL FIELD OF THE INVENTION

The present invention relates generally to pooling resources to accomplish a medical task. More specifically, the present invention relates to anonymizing and queueing medical data for the purpose of being reviewed by any of a number of reviewers who are qualified to review such data. After review the data is restored to its original form to have results communicated to the client.

### BACKGROUND OF THE INVENTION

With the huge increase in the number and sophistication of devices that can remotely monitor medical data comes an increased need to review this data. To meet this growing demand requires more and more people. It is impractical for medical offices to hire the people needed. Providing a pool of reviewers that can be used by any number of medical offices helps alleviate this problem.

### Definitions

Unless stated to the contrary, for the purposes of the present disclosure, the following terms shall have the following definitions:

Administrators, commonly known as admins or sysops (system operators), are software or system users who have been granted the technical ability to perform certain special actions.

“Application software” is a set of one or more programs designed to carry out operations for a specific application. Application software cannot run on itself but is dependent on system software to execute. Examples of application software include MS Word, MS Excel, a console game, a library management system, a spreadsheet system etc. The term is used to distinguish such software from another type of computer program referred to as system software, which manages and integrates a computer's capabilities but does not directly perform tasks that benefit the user. The system software serves the application, which in turn serves the user.

The term “app” is a shortening of the term “application software”. It has become very popular and in 2010 was listed as “Word of the Year” by the American Dialect Society

“Apps” are usually available through application distribution platforms, which began appearing in 2008 and are typically operated by the owner of the mobile operating system. Some apps are free, while others must be bought. Usually, they are downloaded from the platform to a target device, but sometimes they can be downloaded to laptops or desktop computers.

“API” In computer programming, an application programming interface (API) is a set of routines, protocols, and tools for building software applications. An API expresses a software component in terms of its operations, inputs, outputs, and underlying types. An API defines functionalities that are independent of their respective implementations, which allows definitions and implementations to vary without compromising each other.

A client is a piece of computer hardware or software that accesses a service made available by a server. The server is often (but not always) on another computer system, in which case the client accesses the service by way of a network. The term applies to programs or devices that are part of a client-server model.

“Electronic Mobile Device” is defined as any computer, phone, smartphone, tablet, or computing device that is comprised of a battery, display, circuit board, and processor that is capable of processing or executing software. Examples of electronic mobile devices are smartphones, laptop computers, and tablet PCs.

A gateway is a link between two computer programs or systems. A gateway acts as a portal between two programs allowing them to share information by communicating between protocols on a computer or between dissimilar computers.

“GUI”. In computing, a graphical user interface (GUI) sometimes pronounced “gooey” (or “gee-you-eye”)) is a type of interface that allows users to interact with electronic devices through graphical icons and visual indicators such as secondary notation, as opposed to text-based interfaces, typed command labels or text navigation. GUIs were introduced in reaction to the perceived steep learning curve of command-line interfaces (CLIs), which require commands to be typed on the keyboard.

The Hypertext Transfer Protocol (HTTP) is an application protocol for distributed, collaborative, hypermedia information systems. HTTP is the foundation of data communication for the World Wide Web. Hypertext is structured text that uses logical links (hyperlinks) between nodes containing text. HTTP is the protocol to exchange or transfer hypertext.

The internet of things, or IoT, is a system of interrelated computing devices, mechanical and digital machines, objects, animals or people that are provided with unique identifiers (UIDs) and the ability to transfer data over a network without requiring human-to-human or human-to-computer interaction.

The Internet Protocol (IP) is the principal communications protocol in the Internet protocol suite for relaying datagrams across network boundaries. Its routing function enables internetworking, and essentially establishes the Internet.

An Internet Protocol address (IP address) is a numerical label assigned to each device (e.g., computer, printer) participating in a computer network that uses the Internet Protocol for communication. An IP address serves two principal functions: host or network interface identification and location addressing.

An Internet service provider (ISP) is an organization that provides services for accessing, using, or participating in the Internet.

iOS (originally iPhone OS) is a mobile operating system created and developed by Apple Inc. and distributed exclusively for Apple hardware. It is the operating system that presently powers many of the company's mobile devices, including the iPhone, iPad, and iPod touch.

A “mobile app” is a computer program designed to run on smartphones, tablet computers and other mobile devices, which the Applicant/Inventor refers to generically as “a computing device”, which is not intended to be all inclusive of all computers and mobile devices that are capable of executing software applications.

A “mobile device” is a generic term used to refer to a variety of devices that allow people to access data and information from wherever they are. This includes cell phones and other portable devices such as, but not limited to, PDAs, Pads, smartphones, and laptop computers.

A “module” in software is a part of a program. Programs are composed of one or more independently developed modules that are not combined until the program is linked. A single module can contain one or several routines or steps.

A “module” in hardware, is a self-contained component.

An operating system (OS) is software that manages computer hardware and software resources and provides common services for computer programs. The operating system is an essential component of the system software in a computer system. Application programs usually require an operating system to function.

Push Notification, Push, or server push describes a style of Internet-based communication where the request for a given transaction is initiated by the publisher or central server. It is contrasted with pull/get, where the request for the transmission of information is initiated by the receiver or client.

A server is a running instance of an application (software) capable of accepting requests from the client and giving responses accordingly. Servers can run on any computer including dedicated computers, which individually are also often referred to as “the server”.

“SMS” (short message service) is a text messaging service component of most telephone, internet, and mobile-device systems. It uses standardized communication protocols to enable mobile devices to exchange short text messages.

A “software application” is a program or group of programs designed for end users. Application software can be divided into two general classes: systems software and applications software. Systems software consists of low-level programs that interact with the computer at a very basic level. This includes operating systems, compilers, and utilities for managing computer resources. In contrast, applications software (also called end-user programs) includes database programs, word processors, and spreadsheets. Figuratively speaking, applications software sits on top of systems software because it is unable to run without the operating system and system utilities.

A “software module” is a file that contains instructions. “Module” implies a single executable file that is only a part of the application, such as a DLL. When referring to an entire program, the terms “application” and “software program” are typically used. A software module is defined as a series of process steps stored in an electronic memory of an electronic device and executed by the processor of an electronic device such as a computer, pad, smart phone, or other equivalent device known in the prior art.

A “software application module” is a program or group of programs designed for end users that contains one or more files that contains instructions to be executed by a computer or other equivalent device.

A “smartphone” (or smart phone) is a mobile phone with more advanced computing capability and connectivity than basic feature phones. Smartphones typically include the features of a phone with those of another popular consumer device, such as a personal digital assistant, a media player, a digital camera, and/or a GPS navigation unit. Later smartphones include all of those plus the features of a touchscreen computer, including web browsing, wideband network radio (e.g. LTE), Wi-Fi, 3rd-party apps, motion sensor and mobile payment.

URL is an abbreviation of Uniform Resource Locator (URL), it is the global address of documents and other resources on the World Wide Web (also referred to as the “Internet”).

A “User” is any person registered to use the computer system executing the method of the present invention.

In computing, a “user agent” or “useragent” is software (a software agent) that is acting on behalf of a user. For example, an email reader is a mail user agent, and in the Session Initiation Protocol (SIP), the term user agent refers to both end points of a communications session. In many cases, a user agent acts as a client in a network protocol used in communications within a client-server distributed computing system. In particular, the Hypertext Transfer Protocol (HTTP) identifies the client software originating the request, using a “User-Agent” header, even when the client is not operated by a user. The SIP protocol (based on HTTP) followed this usage.

A “web application” or “web app” is any application software that runs in a web browser and is created in a browser-supported programming language (such as the combination of JavaScript, HTML and CSS) and relies on a web browser to render the application.

A “website”, also written as Web site, web site, or simply site, is a collection of related web pages containing images, videos or other digital assets. A website is hosted on at least one web server, accessible via a network such as the Internet or a private local area network through an Internet address known as a Uniform Resource Locator (URL). All publicly accessible websites collectively constitute the World Wide Web.

A “web page”, also written as webpage is a document, typically written in plain text interspersed with formatting instructions of Hypertext Markup Language (HTML, XHTML). A web page may incorporate elements from other websites with suitable markup anchors.

Web pages are accessed and transported with the Hypertext Transfer Protocol (HTTP), which may optionally employ encryption (HTTP Secure, HTTPS) to provide security and privacy for the user of the web page content. The user's application, often a web browser displayed on a computer, renders the page content according to its HTML markup instructions onto a display terminal. The pages of a website can usually be accessed from a simple Uniform Resource Locator (URL) called the homepage. The URLs of the pages organize them into a hierarchy, although hyperlinking between them conveys the reader's perceived site structure and guides the reader's navigation of the site.

### SUMMARY OF THE INVENTION

Data is collected from any number of Remote Patient Monitoring devices and is stored in a HIPAA certified database. This data can be linked to a person at this point. To analyze the data, the links to the patients are anonymized so that the data now has no Personally Identifiable Information (PII). This data is placed in a database in a manner that allows a group of medical analysts to select any number of records to analyze. Each record is also being analyzed by Artificial Intelligence (AI). Both the AI and human analyst data are stored with the anonymized record. If the results match, the records are transmitted back to the original database and are rejoined with the patient data. If the results do not match, they will be sent to another database where another medical analyst can review the data. After a second human review, the results are transmitted back to the original database and are rejoined with the patient data.

Once the data is returned and rejoined with the patient data, the healthcare provider who looks after the patient is informed that results are ready. At this point, either the provider or the provider's staff, or a small pool of “patient contactors” who are certified to review personally identifiable patient data, will contact the patient with information about their test results. This contact is crucial to ensure the patient receives timely information about any medical conditions they may have and what their treatment options may be.

### BRIEF DESCRIPTION OF THE DRAWINGS

The accompanying drawings, which are incorporated herein form a part of the specification, illustrate the present invention and, together with the description, further explain the principles of the invention and to enable a person skilled in the pertinent art to make and use the invention.

FIG. 1 is flow chart illustrating the high-level logic view of the system of the present invention for collecting, analyzing, and processing medical data.

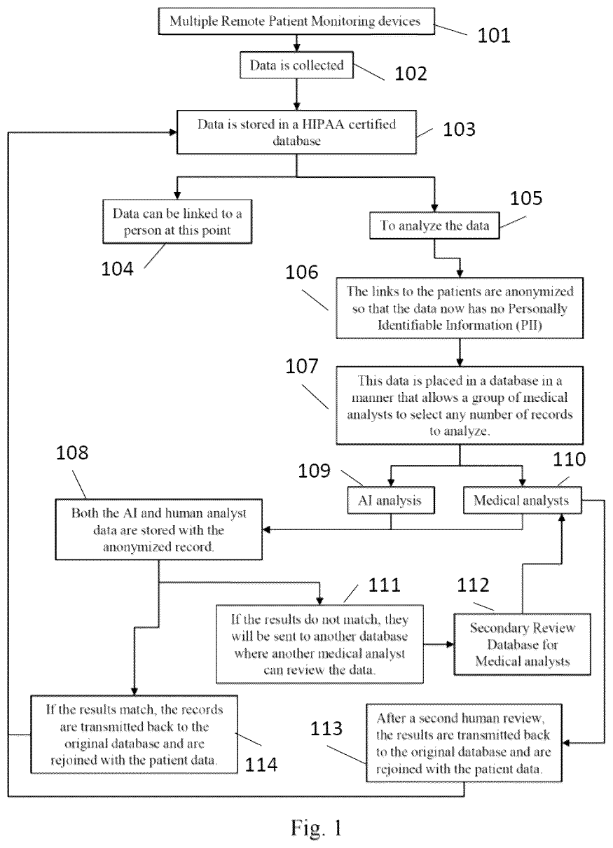

FIG. 2 is a flow chart illustrating the process taught by the present invention after the data is returned and rejoined with the patient data after being analyzed by the AI, and at least one medical analyst.

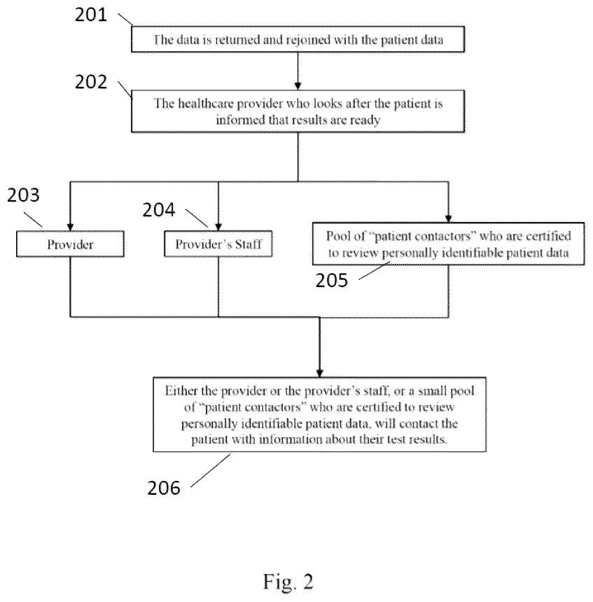

FIG. 3 is a flow chart illustrating how the data is gathered, sorted, and queued for review by the AI/machine and a human reviewer.

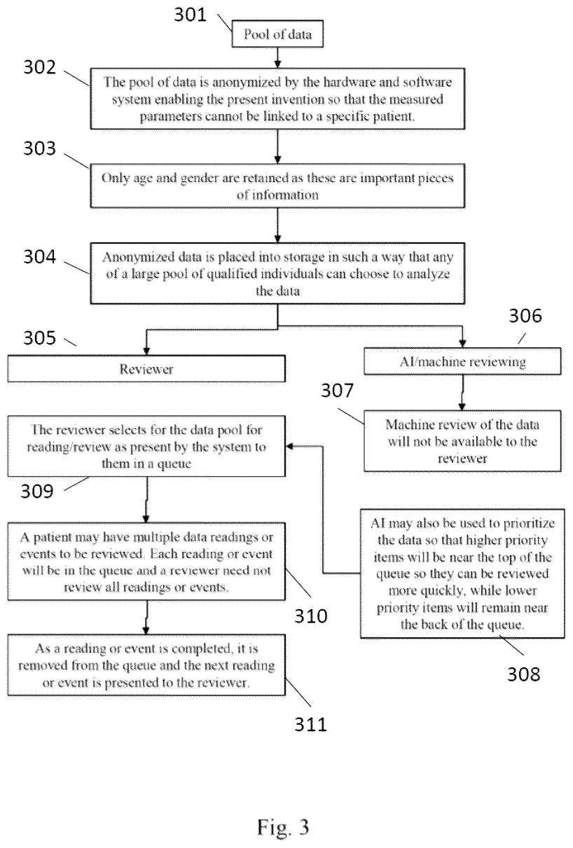

FIG. 4 is a flow chart explain the method/process of creating reports and providing them to a health care provider or staff for retrieval and patent contact/follow up.

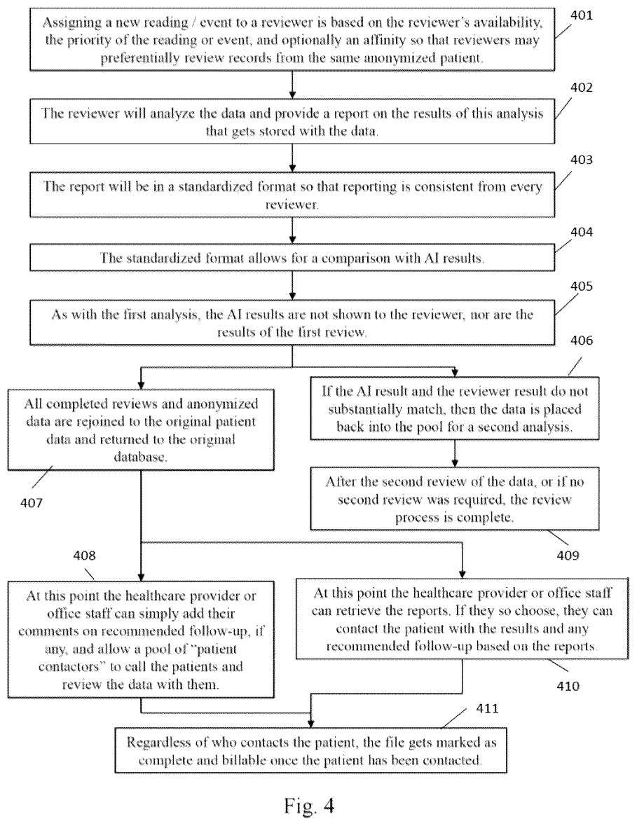

### DETAILED DESCRIPTION OF THE INVENTION

The present invention is a process for caring for patients using remote patient monitoring. The remote monitoring is not part of this process, but the analysis of the data and subsequent report to the patient is part of the process. The challenge faced by most healthcare providers when working with remote patient monitoring is not having the time or resources to analyze the data from many patients.

The present invention will allow analysis to be completed by qualified individuals who do not need personally identifiable information about the patient. Many healthcare providers will have many patients each that require remote patient monitoring of one or more medical parameters. This creates a large amount of data that would be nearly impossible for a healthcare provider or staff to analyze.

Data is collected 102 from any number of Remote Patient Monitoring devices 101 and is stored in a HIPAA certified database 103. This data can be linked to a person at this point 104. To analyze the data 105, the links to the patients are anonymized so that the data now has no Personally Identifiable Information (PII) 106. This data is placed in a database in a manner that allows a group of medical analysts to select any number of records to analyze 107. Each record is also being analyzed by Artificial Intelligence (AI) 109. Both the AI and human analyst data are stored with the anonymized record 108. If the results match, the records are transmitted back to the original database and are rejoined with the patient data 114. If the results do not match, they will be sent to another database 112 where another medical analyst 110 can review the data 111. After a second human review, the results are transmitted back to the original database and are rejoined with the patient data 113.

Once the data is returned and rejoined with the patient data 201, the healthcare provider who looks after the patient is informed that results are ready 202. At this point, either the provider 203 or the provider's staff 204, or a small pool of “patient contactors” who are certified to review personally identifiable patient data 205, will contact the patient with information about their test results 206. This contact is crucial to ensure the patient receives timely information about any medical conditions they may have and what their treatment options may be.

The present invention takes this large pool of data 301 and anonymizes it so that the measured parameters cannot be linked to a specific patient 302. Only age and gender are retained as these are important pieces of information 303.

After anonymization, Artificial Intelligence (AI) is applied to the data to form a machine review 306. This anonymized data is placed into storage in such a way that any of a large pool of qualified individuals can choose to analyze the data 304, but the machine review of the data will not be available to the reviewer 307. AI may also be used to prioritize the data so that higher priority items will be near the top of the queue so they can be reviewed more quickly, while lower priority items will remain near the back of the queue 308.

A patient may have multiple data readings or events to be reviewed. Each reading or event will be in the queue and a reviewer need not review all readings or events 310. As a reading or event is completed, it is removed from the queue and the next reading or event is presented to the reviewer 311.

The mechanism for assigning a new reading/event to a reviewer 305 is based on the reviewer's availability, the priority of the reading or event, and optionally an affinity so that reviewers may preferentially review records from the same anonymized patient 401.

The reviewer will analyze the data and provide a report on the results of this analysis that gets stored with the data 402. The report will be in a standardized format so that reporting is consistent from every reviewer 403. In addition, a standardized format allows for a comparison with AI results 404.

If the AI result and the reviewer result do not substantially match, then the data is placed back into the pool for a second analysis 406. As with the first analysis, the AI results are not shown to the reviewer, nor are the results of the first review 405. After the second review of the data, or if no second review was required, the review process is complete 409.

All completed reviews and anonymized data are rejoined to the original patient data and returned to the original database 407. At this point the healthcare provider or office staff can retrieve the reports. If they so choose, they can contact the patient with the results and any recommended follow-up based on the reports 410. Or they can simply add their comments on recommended follow-up, if any, and allow a pool of “patient contactors” to call the patients and review the data with them 408. Regardless of who contacts the patient, the file gets marked as complete and billable once the patient has been contacted 411.

The system is set to run on a computing device or mobile electronic device. A computing device or mobile electronic device on which the present invention can run would be comprised of a CPU, storage device, keyboard, monitor or screen, CPU main memory and a portion of main memory where the system resides and executes. Any general-purpose computer, smartphone, or other mobile electronic device with an appropriate amount of storage space is suitable for this purpose. Computer and mobile electronic devices like these are well known in the art and are not pertinent to the invention. The system can also be written in several different languages and run on a number of different operating systems and platforms.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

The embodiments of the invention in which an exclusive property or privilege is claimed are defined as follows:

1. A method for analyzing patient data from remote patient monitoring, recorded on computer-readable medium and capable of execution by a computer, the method comprising the steps of:
providing one or more remote patient monitoring devices;
collecting remote patient data by the one or more remote patient monitoring devices;
storing the collected data in a secure database;
anonymizing the data;
the anonymized data is placed into a secure storage area;
subjecting the data to Artificial Intelligence (AI) as a first analysis;
prioritizing the data using AI in the queue so that higher priority items are at the top of the queue;
providing access to the data to a pool of analysts with proper credentials to perform an analysis;
completing data analysis;
creating a standardized report;
storing the standardized report with the data following completion of the analysis;
if the report and the AI are not in substantial agreement, the data is placed back in the pool for additional analysis; and
the data, along with AI and human report(s) are rejoined with the patient data and restored to the original server.

2. The method of claim 1, wherein
the data is anonymized, along with gender and age.

3. The method of claim 1, wherein
the data analysis is completed by either AI analysis, medical analysis, or a combination of both.

4. The method of claim 1, wherein
the healthcare provider or staff will add any comments or patient follow-up if desired.

5. The method of claim 4, wherein
the healthcare provider staff or a pool of “patient contactors” will contact the patients with results and follow-up.

6. The method of claim 5, wherein
after contact has been made with the patient, the file will be marked as complete and billable.

7. The method of claim 6, wherein
the people reviewing the data can use an app and swipe right if data is within normal parameters, and swipe left when they are not.

8. The method of claim 7, wherein
a separate queue will be created for patients that do not transmit their required medical data which will send out contact with the patient to remind them to measure the missing data.

9. The method of claim 8, wherein
providers can rate their patients regarding compliance in providing data in a timely manner.

10. A computer-based method for analyzing patient data from a remote patient monitoring device, the method-comprising the steps of:
providing on and executing computer-readable media for analyzing patient data from a remote patient monitoring device, by a computer machine;
providing one or more remote patient monitoring devices;
collecting remote patient data by the one or more remote patient monitoring devices;
storing the collected data in a secure database;
linking the data to a person;
anonymizing the data;
placing the data in a database;
selecting data to analyze;
analyzing the data;
analyzing the data by Artificial Intelligence (AI);
receiving a human analysis of the data;
comparing the results of the AI analysis and the human analysis;
if the results match, the data is transmitted back to the database and are rejoined with the patient data; or
if the results do not match, the data is sent to a second database where another medical analyst can review the data; and
after a second human review, all data is returned to the database and rejoined with the patient data.

11. The method of claim 10, wherein
the links to the patients are anonymized, creating an anonymized record, so that the data now has no Personally Identifiable Information (PII).

12. The method of claim 11, wherein
both the AI and human analyst data are stored with the anonymized record.

13. The method of claim 10, wherein
informing the healthcare provider who looks after the patient that results are ready once the data is returned and rejoined with the patient data; and
contacting the patient with information about their test results either the provider or the provider's staff, or a small pool of “patient contactors” who are certified to review personally identifiable patient data.

14. The method of claim 10, further comprising the steps of
anonymizing a pool of data it so that one or more measured parameters cannot be linked to a specific patient; and
measured parameters including age and gender are retained.

15. The method of claim 14, wherein
after anonymization, Artificial Intelligence (AI) is applied to the data to form a machine review; and
this anonymized data is placed into storage so that any of a large pool of qualified individuals can choose to analyze the data, but the machine review of the data will not be available to the reviewer.

16. The method of claim 10, further comprising the steps of
AI is used to prioritize the data so that higher priority items are at the top of a queue, while lower priority items will remain near a back of the queue.

17. The method of claim 10, wherein
a patient may have multiple data readings or events to be reviewed;
each reading or event will be in a queue and a reviewer need not review all readings or events; and
as a reading or event is completed, it is removed from the queue and the next reading or event is presented to the reviewer.

18. The method of claim 17, wherein
assigning a new reading/event to a reviewer is based on the reviewer's availability, the priority of the reading or event, and optionally an affinity so that reviewers may preferentially review records from the same anonymized patient;
the reviewer will analyze the data and provide a report on the results of this analysis that gets stored with the data; and
the report will be in a standardized format so that reporting is consistent from every reviewer and a standardized format allows for a comparison with AI results.

19. The method of claim 18, wherein
if the AI result and the reviewer result do not substantially match, then the data is placed back into a pool for a second analysis;
the AI results are not shown to the reviewer, nor are the results of the first review; and
after the second review of the data, or if no second review was required, the review process is complete.

20. The method of claim 19, wherein
all completed reviews and anonymized data are rejoined to the original patient data and returned to the original database;
a healthcare provider or office staff can retrieve the reports;
if they so choose, they can contact the patient with the results and any recommended follow-up based on the reports; or
they can simply add their comments on recommended follow-up, if any, and allow a pool of “patient contactors” to call the patients and review the data with them; and
regardless of who contacts the patient, a file gets marked as complete and billable once the patient has been contacted.
